(19) United States
(12) Patent Application Publication
(10) Pub. No.: US 2005/0154272 A1
(43) Pub. Date: Jul. 14, 2005

(54) Method and device for monitoring analyte concentration by use of differential osmotic pressure measurement

(75) Inventors: Holger Dirac, Birkeroed (DK); Kasper Oktavio Schweitz, Hilleroed (DK)

Correspondence Address: MCCORMICK, PAULDING & HUBER LLP, CITY PLACE II, 185 ASYLUM STREET, HARTFORD, CT, 06103, US

(73) Assignee: Danfoss A/S, Nordborg (DK)

(21) Appl. No.: 10/501,746

(22) PCT Filed: Jan. 21, 2003
(86) PCT No.: PCT/DK03/00036

(30) Foreign Application Priority Data
Jan. 23, 2002 (DK) PA 2002 00120

(51) Int. Cl. A61B 5/00

(52) U.S. Cl. 600/365; 600/309; 600/561

(57) ABSTRACT

A method is provided for the determination of the concentration of compounds in body tissue and fluids. The method utilises two compartments containing reference solutions, which are separated from the sample by two different semi-permeable membranes, in a serial manner, whereby a difference in osmotic pressure occurs in the two compartments due to compounds, which can permeate one of the membranes, but not the other. The difference in osmotic pressure reflects the concentration of these compounds. The method is especially suited for analysis of the concentration of glucose in blood or tissue of diabetic patients, where a device is implanted underneath the skin of the patient and where the method is carried out by using the implanted device.

### CROSS-REFERENCE TO RELATED APPLICATIONS

This application is entitled to the benefit of and incorporates by reference essential subject matter disclosed in International Application No. PCT/DK03/00036 filed on Jan. 21, 2003 and Danish Patent Application No. PA 2002 00120 filed on Jan. 23, 2002.

### FIELD OF THE INVENTION

This invention relates to biological sensors, more specifically to implantable sensors for monitoring species such as glucose, in a living creature, for example, in the human or animal body. Further specifically, but not exclusively, this invention relates to biological sensors for the detection of glucose in blood or tissue of a diabetic patient.

### BACKGROUND OF THE INVENTION

Diabetic patients can improve their life quality expectancy by maintaining their blood glucose concentration close to the natural level of a healthy person. To achieve this natural concentration, diabetes patients must frequently measure their glucose concentration, and adjust their insulin dosing in accordance with the measured concentration. Usually, a blood sample is obtained for measurement of blood glucose concentration, and there are a number of different glucose test kits on the market based on measurement on blood samples. The disadvantage of these test kits is the need to take a blood sample, which must be collected from a suitable place on the body.

Self-monitoring devices, based on capillary blood glucose, are practical but still require repeated and frequent skin punctures, which is inconvenient for the patient, and requires certain hygienic precautions.

Biological sensors in the form of implantable devices are also known in the art and include electrochemical devices and optical devices based on the creation of an electrical or optical signal by the consumption of the compound detected by the analysis. An example is U.S. Pat. No. 6,011,984, which discloses methods utilising an amplification component. The sensitivity and the responsivity of such devices are influenced by the formation of a bio film, for example, by fibrous encapsulation of the device, which reduces the transport rate of the compound to the sensor. Depending on the specific sensor, other mechanisms which cause deterioration of the sensor performance of implanted devices, may also be present, for example, membrane de-lamination and degradation, enzyme degradation and electrode passivation.

U.S. Pat. No. 5,337,747 discloses an implantable device comprising two measurement chambers each of which comprises an internal measurement chamber isolated from its surroundings by a glucose-impermeable membrane for the first measurement chamber, and by a glucose-permeable membrane which is impermeable to molecules larger than glucose for the second measurement chamber. Each measurement chamber is connected to a pressure sensor and linked to an electronic system provided for informing the environment outside the organism of the value of the pressure measured in each of the two measurement chambers. The pressure difference between the two measurement chambers is interpreted as the osmotic pressure, and this pressure will correspond to a specific level of glucose.

However, the two chambers that constitute the implantable device of U.S. Pat. No. 5,337,747 are in contact with the surroundings at two different locations due to their side-by-side arrangement. This might result in significant detection errors in cases where the conditions (level of glucose, bio fouling tendency etc.) are different at the two locations. Another problem is the possibly increased tendency for bio fouling of the glucose-impermeable membrane as compared to the glucose-permeable membrane. This increased tendency for bio fouling will change the transport characteristics of the glucose-impermeable membrane and, thus, increase the need for frequent re-calibration of the device, or replacement of the device.

It is an object of this invention to overcome the problems with inhomogeneity, and to reduce the rate of bio fouling of the glucose-impermeable membrane.

### SUMMARY OF THE INVENTION

As would be obvious to those skilled in the art, the measurement principle disclosed with this invention is not limited to implanted devices in diabetic patients for measuring glucose concentration, but could be used in many other applications. The basic idea is used for measuring species in locations which are difficult to access, and where the physical- and chemical conditions vary over time. This could be the measurement of the glucose concentration in a bioreactor or in fruit juice etc.

The object of this invention is achieved by having two compartments, one of them at least partially defined to the exterior by a first set of barriers permeable for a set of species, the other compartment separated from the first compartment by a second set of barriers permeable only for a subset of the species, only a subset of species that permeates into the first compartment permeates further on into the other compartment. Hereby is achieved that the membranes are connected in a serial manner and, thus, only the glucose-permeable membrane is exposed to bio fouling from species in the surroundings, which cannot permeate through the first set of barriers. Furthermore, the serial arrangement of the membranes alleviates the problems due to inhomogeneity, as only one compartment is exposed to the surroundings.

In one embodiment of the invention, the permeability of the two sets of barriers cause a specific species to be able to permeate into the first compartment, but not into the other compartment. This is achieved in that the first set of barriers is permeable for species up to and including the size of a specific molecule, and the second set of barriers is permeable for species below the size of same specific molecule.

In another embodiment of the invention, some of the compartments are filled with a known concentration of species, unable to permeate through the barrier defining the compartment. Hereby is achieved that these compartments work as reference compartments, the determination of the concentration of a specific species occurring through comparison with the reference compartments.

In a specific embodiment of the invention the permeability of the two sets of barriers is such that glucose will be able to permeate into one of the compartments, but not into the other compartment. Hereby a sensor specific for detecting the concentration of glucose in a sample is achieved.

In another embodiment of the invention, the pressure difference between the two compartments is detected, so that a value corresponding to the concentration of species permeating into one of the compartments, but not into the other, is obtained.

In a more specific embodiment of the invention a separate pressure sensor detects the pressure exterior to the two compartments. The influence of pressure variations due to conditions external to the device can hereby be compensated.

In another specific embodiment of the invention, the pressure sensing is at least partly formed as a deflection measurement of a flexible compartment, which will increase or decrease in volume when the pressure in the compartment increases or decreases.

### BRIEF DESCRIPTION OF THE DRAWINGS

In the following the invention is described in detail with reference to the drawings showing:

FIG. 1 shows a principal embodiment of the invention showing two compartments, each with a separate barrier.

FIG. 2 shows a principal embodiment of a device, where one of the compartments is divided into multiple reference compartments.

FIG. 3 shows a principal embodiment of a device having a structure in the form of a disc.

FIG. 4 shows an exploded view of the principal device of FIG. 3.

FIG. 5 shows an exploded view of the principal device of FIG. 3, where the barriers are supported by a mechanical structure.

FIG. 6 is a diagram with simulation results for the performance of a device.

### DETAILED DESCRIPTION OF THE PREFERRED EMBODIMENTS

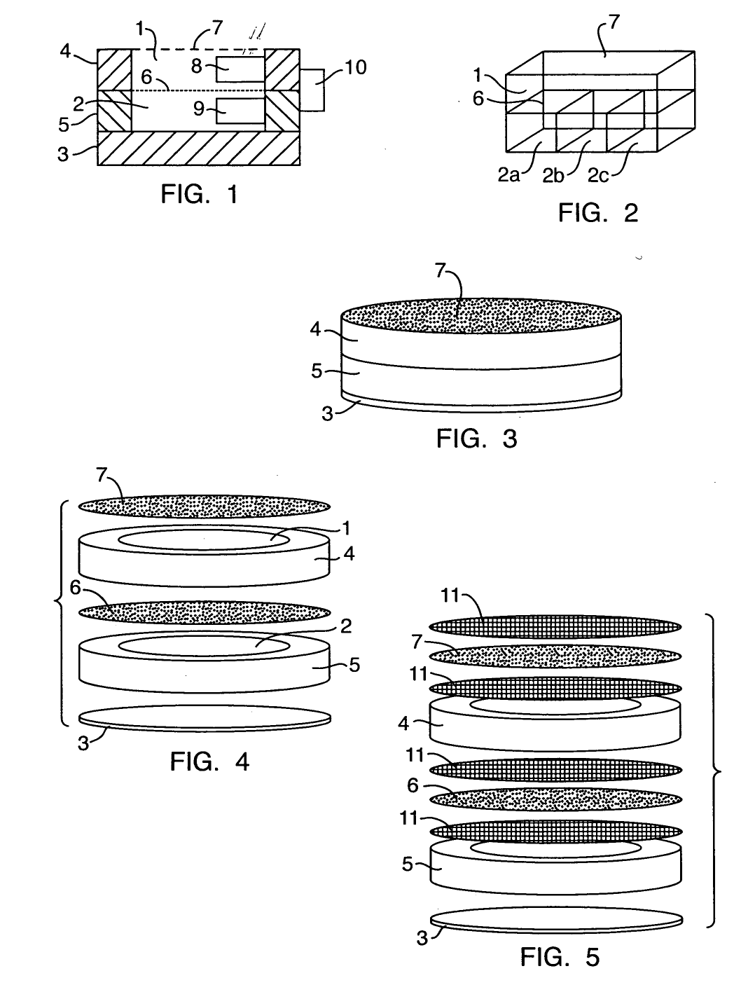

FIG. 1 shows a sectional view of a device, where two compartments 1 and 2 are stacked on a base plate 3. A perspective view of the same device is shown in FIG. 3. The device is implantable into the human body, and is suitable for detecting the glucose level in blood or interstitial fluid.

Compartment 1 is sealed to the exterior by the ring member 4 and by a barrier 7, and compartment 2 is sealed to the exterior by ring member 5 and base plate 3. A barrier 6 seals the two compartments 1 and 2 from each other.

Membranes with a specific Molecular Weight Cut Off (MWCO) form each of the barriers 6 and 7. The membrane forming the barrier 6 has an MWCO just below the size of the glucose molecule, and the membrane forming the barrier 7 has an MWCO just above the size of the glucose molecule. This means, that only species with the size of the glucose molecule or below will penetrate from the exterior into compartment 1, and that only species with a size below the glucose molecule will penetrate from compartment 1 into compartment 2. The osmotic pressure will then appear over the membrane forming the barrier 6, between the two compartments, and two independent pressure sensors 8 and 9 detect the pressure in each compartment. The two pressure sensors 8 and 9 could be substituted with a differential pressure sensor, which is capable of detecting the pressure difference between the two compartments 1 and 2. An additional pressure sensor 10 is for the purpose of detecting the surrounding pressure, e.g. the pulse beat and exterior pressure variations can thus be taken into account.

FIG. 4 shows an exploded view of FIG. 3, the ring shape of each element 3-7 becoming more visible. The volume of compartment 1 is formed by the internal diameter of the ring shaped element 4 and by the height of element 4. Similarly, the volume of compartment 2 is formed by the internal diameter of the ring shaped element 5 and by the height of element 5.

For glucose measurement in a human body, the device of FIG. 4 could consist of ring shaped elements 4 and 5 with an inner diameter of 500 μm and a height of 240 μm. The membrane 7 could be a 500 Da Biotech Cellulose Ester Membrane from Spectrum Laboratories Inc., meaning that the membrane has an MWCO of 500 g/mol. This size will allow the molecule glucose to permeate the membrane, and hence enter the compartment 1. The membrane 6 could be a 100 Da Biotech Cellulose Ester Membrane from Spectrum Laboratories Inc., meaning that the membrane has an MWCO of 100 g/mol. This size will prevent glucose from permeating the membrane.

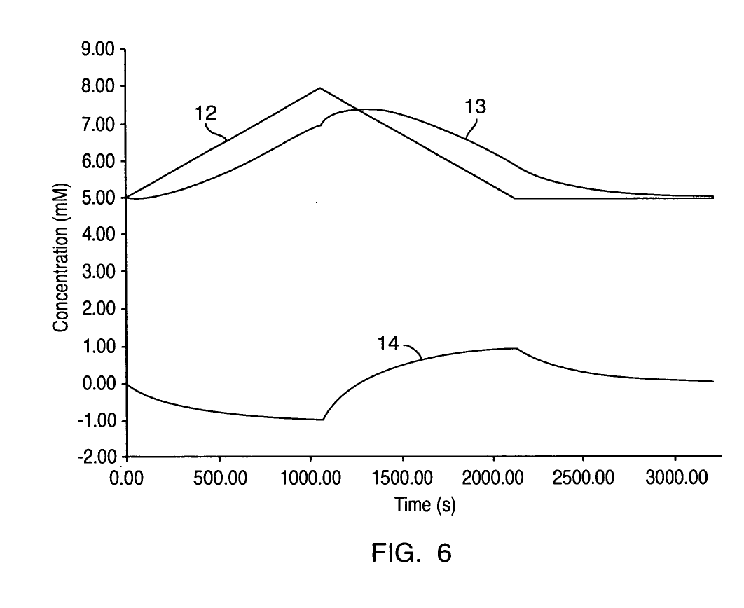

With dimensions and specifications as described above, the device of FIG. 4 will perform as indicated in FIG. 6. Curve 12 shows the glucose concentration in blood or interstitial fluid, varying with the highest rate possible in the human body. Curve 13 indicates the glucose level as given by the device, and curve 14 is the difference between the actual glucose level and the detected glucose level. The difference is within +1 mM, which is regarded as an acceptable deviation.

FIG. 5 shows a device similar to that of FIG. 4, only with a rigid element 11 on either side of each membrane. The element 11 must have no influence on the MWCO of the membranes. The purpose of this rigid element is to minimise the deflection of the membrane, due to the pressure difference across it. With less deflection of the membranes, the volume of the compartments will be less dependent of the pressure differences, and less amount of species has to permeate through the membranes to yield the equilibrium pressure (osmotic pressure), which makes the response time of the device shorter and, thus, the device more accurate. The pressure sensors can be used as previously described. The pressure sensors used in the device shown in FIGS. 4 and 5 might be substituted with a deflection sensor, where the deflection of the membrane then corresponds to the concentration of a given compound.

FIG. 2 shows a device in a 3D-view, where the bottom compartment 2 is divided into a number of compartments (2a, 2b and 2c). The top compartment 1 is defined to the exterior by a membrane 7 and to the bottom compartment by a membrane 6, in the same way as described for FIG. 1. The bottom part itself is divided into a number of compartments, here three, each containing a different and known concentration of a given compound. In the case of detecting the level of glucose in a human body, the compound in each of the compartments 2a-c could be glucose, and the membrane 6 should have an MWCO below the size of the glucose molecule.

As there are different concentrations in each compartment, 2a, 2b and 2c, the differential pressure between compartment 1 and each of 2 will vary from each other. By determining which of the compartments 2 has a pressure lower than that of compartment 1, and which one has a pressure higher than that of compartment 1, the compartment 2 with a pressure equal or close to that of compartment 1 can be determined, and hence the concentration of the given compound in compartment 1. The pressure sensor can hereby be substituted with a simple qualitative pressure detector, only capable of detecting the direction of a pressure difference.

As the device can be used as an implantable device, the device may then be powered and data collected from an external device. For this purpose established techniques for biomedical telemetry can be used, e.g. inductively coupled load shift keying or LC resonance frequency modulation. The signal can also be transferred optically using infrared light, e.g. by modulation of an infrared LED or laser diode, or by imaging the inflation/deflation of flexible compartments of the implanted device according to the difference in pressure of the compartments and the external tissue and fluids.

1-10. (canceled)

11. A device comprising at least two compartments, one of said at least two compartments being at least partially defined to an exterior by a first set of barriers permeable for a set of species, another of said at least two compartments being separated from said one of said at lest two compartments by a second set of barriers permeable only for a subset of said species; and wherein only a subset of species that permeates into said one compartment permeates further on into said other compartment(s).

12. A device in accordance with claim 11, wherein said first set of barriers is permeable for species up to and including the size of a specific molecule, and said second set of barriers is permeable for species below said size of said specific molecule.

13. A device in accordance with claim 11, wherein at least one of said compartments is filled with a solution having a known concentration of solutes, all solutes in said solution being unable to permeate through said barriers defining said compartment.

14. A device in accordance with claim 12, wherein said specific molecule is glucose.

15. A device in accordance with claim 13, wherein one of said solutes in said solution is glucose.

16. A device in accordance with claim 11, wherein said device is implantable and has an outer surface, which is substantially biocompatible.

17. A device in accordance with claim 11, wherein said device further contains pressure-sensing means capable of sensing said pressures in said compartments.

18. A device in accordance with claim 17, wherein said pressure sensing means comprises a pressure sensor for sensing said pressure exterior to said compartments.

19. A device in accordance with claim 17, wherein said pressure sensing means contains a differential pressure sensor.

20. A device in accordance with claim 17, wherein said pressure sensing means is at least partly formed by a flexible compartment wherein deflecting of said flexible compartment will, increase or decrease in volume when said pressure in said compartment increases or decreases.
